SEQUENCE LISTING (28 sequences)

SEQ ID NO: 1 - DNA, 22 bp, Actinobacillus actinomycetemcomitans
caggtaagtacttgtacttatg

SEQ ID NO: 2 - DNA, 17 bp, Actinobacillus actinomycetemcomitans
ggattggggtttagccc

SEQ ID NO: 3 - DNA, 21 bp, Actinobacillus actinomycetemcomitans
aggagaaagcttgctttcttg

SEQ ID NO: 4 - DNA, 20 bp, Actinobacillus actinomycetemcomitans
ggataagggttgcgctcgtt

SEQ ID NO: 5 - DNA, 21 bp, Porphyromonas gingivalis
catgatcttagcttgctaagg

SEQ ID NO: 6 - DNA, 22 bp, Prevotella intermedia
cattatgtgcttgcacattctg

SEQ ID NO: 7 - DNA, 20 bp, Bacteroides forsythus
gakggtagcaatacctgtcg

SEQ ID NO: 8 - DNA, 20 bp, Treponema denticola
cggtaagggagagcttgctc

SEQ ID NO: 9 - DNA, 20 bp, Treponema denticola
taagggagagcttgctctcc

SEQ ID NO: 10 - DNA, 18 bp, Treponema denticola
gcaagtcgaacggtaagg

SEQ ID NO: 11 - DNA, 19 bp, Unknown
  Oligonucleotide derived from periodontitis/caries associated bacterium
ctgctgcctcccgtaggag

SEQ ID NO: 12 - DNA, 19 bp, Streptococcus mutans
actgtgcttgcacaccgtg

SEQ ID NO: 13 - DNA, 19 bp, Streptococcus sobrinus
caccggacttgctccagtg

SEQ ID NO: 14 - DNA, 15 bp, Actinobacillus actinomycetemcomitans
cgaagaagaactcag

SEQ ID NO: 15 - DNA, 17 bp, Actinobacillus actinomycetemcomitans
tccgaagaagaactcag

SEQ ID NO: 16 - DNA, 16 bp, Actinobacillus actinomycetemcomitans
cgcgtagaatcgggag

SEQ ID NO: 17 - DNA, 16 bp, Actinobacillus actinomycetemcomitans
cgcgtagggtcgggag

SEQ ID NO: 18 - DNA, 16 bp, Actinobacillus actinomycetemcomitans
cgcgtagagtcgggag

SEQ ID NO: 19 - DNA, 17 bp, Porphyromonas gingivalis
catacacttgtattatt

SEQ ID NO: 20 - DNA, 19 bp, Porphyromonas gingivalis
catacacttgtattattgc

SEQ ID NO: 21 - DNA, 17 bp, Prevotella intermedia
atgttgtccacatatgg

SEQ ID NO: 22 - DNA, 15 bp, Bacteroides forsythus
aacaggggttccgca

SEQ ID NO: 23 - DNA, 20 bp, Treponema denticola
cattyacctttatgtaaatg

SEQ ID NO: 24 - DNA, 20 bp, Treponema denticola
catttacataaaggtraatg

SEQ ID NO: 25 - DNA, 16 bp, Treponema denticola
tgggtgacctgccctg

SEQ ID NO: 26 - DNA, 15 bp, Treponema denticola
cttyaggatgggccc

SEQ ID NO: 27 - DNA, 19 bp, Streptococcus sobrinus
cataagaggagttaactca

SEQ ID NO: 28 - DNA, 17 bp, Streptococcus mutans
ccgcataatattaatta

(19) United States
(12) Patent Application Publication
(10) Pub. No.: US 2005/0019772 A1
(43) Pub. Date: Jan. 27, 2005

(54) Method for detecting bacteria associated with parodontitis and tooth decay

(76) Inventor: Michael Weizenegger, Wiesloch (DE)

Correspondence Address: SHANKS & HERBERT, 1301 K STREET, N.W., SUITE 1100 - EAST TOWER, WASHINGTON, DC, 20005, US

(21) Appl. No.: 10/494,602

(22) PCT Filed: Nov. 5, 2002
(86) PCT No.: PCT/EP02/12334

(30) Foreign Application Priority Data
Nov. 5, 2001 (DE) 101-54-290.9

(51) Int. Cl. C12Q 1/68; C12P 19/34; A61C 5/00

(52) U.S. Cl. 435/6; 433/217.1; 435/91.2

(57) ABSTRACT

The invention relates to the diagnosis of microorganisms, especially the detection of microorganisms which are associated with the disease known as parodontitis or tooth decay. The information relates to hybridization and amplification methods in particular, in addition to coupled amplification/hybridization methods with sequence-specific probes or primers.

The present invention relates to the field of diagnosis of microorganisms, in particular to the detection of bacteria associated with periodontitis and caries disorders. More specifically, the invention relates to hybridization methods and amplification methods and also to coupled amplification/hybridization methods using sequence-specific probes and primers, respectively.

The detection and accurate identification of bacteria play a very important part in periodontology so as to be able to introduce an appropriate treatment.

Periodontitis is an infectious disorder of the tooth-holding apparatus. The transitional zone between the hard tissues of the tooth and the soft tissues of the periodontium provides ideal conditions for microbial infections. Functioning immune defenses protect the periodontium from the damaging action of pathogenic substances secreted by microorganisms. The immunocompetent host is capable of successfully fending off everyday microbial attacks, thus preventing an infection, i.e. propagation in the periodontium. Periodontal inflammation is the local response to toxins released by microorganisms. In the first phase of infection, enzymes and cytotoxic metabolites from microbial plaque and from oral fluid alter the tissue. The tissue immune response comprises a number of mechanisms which, although representing primarily resistance to tissue-destroying substances, result in the destruction of the gingival tissue parts. The three highly periodontitis-associated bacterial species Actinobacillus actinomycetemcomitans, Porphyromonas gingivalis and Bacteriodes forsythus are regarded as being very important for development of periodontitis. Other bacteria such as Campylobacter rectus, Fusobacterium nucleatum, Prevotella intermedius, Eikonella corrodens, Streptococcus intermedius-complex and Treponema denticola are considered to be less highly periodontitis-associated bacteria. Progressing pockets contain large numbers of periodontopathogenic microorganisms, but healthy tissue contains only small amounts thereof, if any. Eliminating the germs or their toxins (e.g. proteases, collagenases, and the like) leads to clinical improvement in the pathology. Therefore microbiological diagnosis plays an important part in therapy planning, in particular when administration of antibiotics is intended. In therapy control too, detection of periodontopathogenic bacteria may sometimes be the only indication of therapeutic success. Another medically important infection of the teeth is caused by sugar-fermenting bacteria. Streptococci, by way of the species Streptococcus mutans and Streptococcus sobrinus, are particularly important here. Due to the formation of sticky sugar polymers, both organisms can adhere well to the smooth dental surfaces and destroy the dentin enamel there by producing acid. This process is moreover aided by the high consumption of sucrose in the industrialized countries.

In recent years, important inventions have been made in order to detect organisms by using very species-specific primers in a nucleic acid amplification reaction. This usually involves detection via gel electrophoresis or via immobilized probes in microtiter plates, similar to the ELISA technique. Unfortunately, these methods are unsuitable when dealing with detecting one or more out of a plurality of possible pathogenic organisms. Bacterial groups of high complexity, large diversity and difficult growth conditions (e.g. strictly anaerobic bacteria), in particular, are very difficult to access by classical culture differentiation and/or delay diagnosis considerably, due to their slow growth. Nucleic acid-based methods distinguished by high specificity and sensitivity are revolutionary here.

It was thus the object of the present invention to provide a highly specific and highly sensitive method for detecting periodontitis- and caries-associated bacteria.

This object was achieved according to the invention by a method for detecting periodontitis- and caries-associated bacteria, which comprises hybridizing under stringent conditions a nucleic acid to be detected which is a fragment of the genome of a periodontitis- or caries-associated bacterium or is complementary to said fragment to a sequence- and/or species-specific nucleic acid probe and subsequently detecting said nucleic acid to be detected or said hybridization of said nucleic acid to be detected to said sequence-specific nucleic acid probe, characterized in that said sequence-specific nucleic acid probe is selected from the sequences with SEQ ID Nos.: 1-28 or is complementary to said sequences, or is a fragment thereof or is complementary to said fragment or comprises any of said sequences or said complementary sequence.

This embodiment of the invention is referred to just as hybridization method hereinbelow. The term hybridization method includes all preferred embodiments.

The term nucleic acid and oligonucleotide means in accordance with the present invention primers, samples, probes and oligomeric fragments which are detected. The term nucleic acid and oligonucleotide is furthermore generic for polydeoxyribonucleotides (comprising 2-deoxy-D-ribose) and for polyribonucleotides (comprising D-ribose) or for any other type of polynucleotide which is an N-glycoside of a purine base or of a pyrimidine base, or of a modified purine base or modified pyrimidine base. Included are also according to the invention PNAS, i.e. polyamides having purine/pyrimidine bases. In accordance with the present invention, the terms nucleic acid and oligonucleotide are not regarded as being different; more specifically, use of said terms is not intended to implicate any distinction with respect to length. Said terms include both double- and single-stranded DNA and double- and single-stranded RNA.

According to the invention, a composition comprising the nucleic acid to be detected or a part thereof is hybridized with one or more probes.

It is possible in principle to determine said nucleic acid to be detected and thus, for example, the bacterial species by hybridization with a single specific probe. However, it is also possible to hybridize said composition comprising said nucleic acid to be detected or a part thereof with more than one probe, thereby increasing the meaningfulness of the method. An accurate profile is then obtained, enabling said nucleic acid to be detected and thus, for example, the bacterial species to be determined very reliably.

The skilled worker appreciates that it is possible, starting from the teaching of the present invention, also to design probes which slightly deviate from the probes of the invention but which nevertheless function. Conceivable probes are thus also those which, compared to the probes of the invention having the sequences SEQ ID No.: 1-28, are extended or truncated by at least one, two or three nucleotides at the 5′ and/or 3′ end. It is likewise conceivable that individual or a few nucleotides of a probe can be replaced with other nucleotides, as long as the specificity of said probe and the melting point of said probe are not altered too much. This includes a modification in which the melting temperature of the modified probe does not deviate too greatly from the melting temperature of the original probe. Said melting temperature is determined following the G(=4° C.)+C(=20 C.) rule. It is obvious to the skilled worker that it is also possible to use, in addition to the usual nucleotides A, G, C and T, modified nucleotides such as inosine, etc. The teaching of the present invention provides for such modifications, starting from the subject matter of the claims.

The term hybridization refers to the formation of duplex structures by two single-stranded nucleic acids, owing to complementary base pairing. Hybridization can take place between complementary nucleic acid strands or between nucleic acid strands which have relatively small mismatched regions. The stability of said nucleic acid duplex is measured by way of the melting temperature Tₘ. The melting temperature Tₘ is the temperature (with defined ionic strength and pH) at which 50% of base pairs are dissociated.

Conditions under which merely fully complementary nucleic acids hybridize are referred to as stringent hybridization conditions. Stringent hybridization conditions are known to the skilled worker (e.g. Sambrook et al., 1085, Molecular Cloning—A Laboratory Manual, Cold Spring Harbor Laboratory, Cold Spring Harbor, N.Y.). In general, stringent conditions are selected so as for the melting temperature to be 5° C. lower than the Tₘ for the specific sequence at defined ionic strength and pH. If said hybridization is carried out under less stringent conditions, sequence mismatches are tolerated. It is possible to control the degree of sequence mismatches by altering the hybridization conditions.

Carrying out the hybridization method is known per se to the skilled worker. Thus, after incubation with the solution which may contain the hybridization partner, the solid phases are usually subjected to stringent conditions in order to remove unspecifically bound nucleic acid molecules. The hybridization may be carried out in a conventional manner on a nylon or nitrocellulose membrane (Sambrook et al., Molecular Cloning, Cold Spring Harbor Laboratory, 1989). The principles mentioned therein can be transferred by the skilled worker to other embodiments.

Carrying out the hybridization under stringent conditions is particularly important for the method of the invention. Stringent means in accordance with the present invention that the detection method allows for unambiguous distinction between a positive reaction and a negative reaction in the reaction field of the strip. Hybridization stringency can be improved by the following measures: Probe structure: via the length of the target sequence-complementary structure of the probe; preference is given to 15 to 20 mers.

Running buffer: the salt content influences stringency. The ionic strength is preferably between 100-500 mM, particularly preferably at 250 mM.

It is furthermore possible to individually adjust and optimize stringency by gently denaturing substances in the running buffer (DMSO, formamide, urea) . Stringency is also influenced by the pH of the running buffer.

The length of the target nucleic acid also plays an important part for the sensitivity of the hybridization. Preference is given to nucleic acid strands of 100-500 base pairs in length. Preferably, the double-stranded target nucleic acid must be denatured prior to hybridization. This is effected usually by basic chemicals or by heating, melting the hydrogen bonds responsible for the double-stranded structure. A preferred basic chemical is NaOH at a concentration of from 0.1 to 0.5 M. Particular preference is given to a concentration of 0.25 M NaOH. Heating an aqueous nucleic acid solution to at least 95° C. and subsequent rapid cooling to 4° C. can likewise produce single-stranded structures. Single-strand amplicons, for example as products of the NASBA reaction, should likewise be denatured prior to hybridization in order to dissolve intramolecular structures. Due to the sensitivity of RNA to high pH, said denaturation may preferably be effected by gently denaturing chemicals such as DMSO or formamide, for example.

Aside from the structures of target sequence and probe, the desired hybridization stringency is determined by the composition of hybridization and stringency washing buffers. The hybridization buffers used are usually aqueous buffers having a salt content of between 0.1 and 0.5 M and a pH of 7.5-8.0. Detergents are used for well moistening the probe-carrying phase. Preference is given to sodium dodecyl sulfate (SDS) at a concentration of 0.1-7%. At the particularly preferred high concentration of 7%, SDS has moreover a beneficial effect on signal/background ratios by suppressing unspecific bindings of the enzyme complex. Preference is given to incubating with a stringency washing buffer after hybridization, which destabilizes the double strand due to lower ionic strength. Hybrids which are not 100% complementary are thus separated again. It is possible by adding chemicals (e.g. tetramethylammonium chloride) which influence the hydrogen bonds of the hybrid to adjust the binding strength of G/C and A/T pairs, this possibly being advantageous in multiplex probe systems.

After hybridization, the extent of said hybridization is determined according to the invention, normally by determining the amount of label bound to a solid phase, said label being bound either to the probes or to the nucleic acid to be detected. Detection reactions and detection methods of this kind are known per se to the skilled worker.

In a preferred embodiment of the method, the sequence-specific nucleic acid probe is selected from the sequences with SEQ ID Nos.: 14-28 or is complementary to said sequences, or is a fragment thereof or is complementary to said fragment or comprises any of said sequences or said complementary sequence.

A preferred embodiment of the method of the invention is characterized in that the nucleic acid to be detected is an amplification product, said amplification having been carried out using sequence-specific amplification primers. Particular preference is given to at least one amplification primer being selected from the sequences with SEQ ID Nos.: 1-28 or being complementary to said sequences, or being a fragment thereof or being complementary to said fragment or comprising any of said sequences or said complementary sequence. Most preference is given to at least one amplification primer being selected from the sequences with SEQ ID Nos.: 1-13 or being complementary to said sequences, or being a fragment thereof or being complementary to said fragment or comprising any of said sequences or said complementary sequence.

The amplification primers should be selected in such a way that the amplification product has good steric conditions in combination with the immobilized probe. Palindromic structures which may result in intramolecular foldings can be avoided by suitable primer selection. In the case of labeling with hapten, the spatial arrangement of said hapten (e.g. biotin) in the probe/target nucleic acid hybrid is important. The hapten should be readily accessible for the antibody-enzyme complex.

One embodiment of the method is characterized in that the nucleic acid probes are immobilized. In this case, it is advantageous if the nucleic acid to be detected is labeled. In another form of the method, the nucleic acid probes are labeled. In this case, it is advantageous if the nucleic acid to be detected is immobilized.

The invention therefore furthermore relates to a method for detecting periodontitis- and caries-associated bacteria, which comprises—amplifying a nucleic acid to be detected which is a fragment of the genome of a periodontitis— or caries associated bacterium or is complementary to said fragment, said amplification being carried out using primers at least one of which has a sequence which is essentially a partial sequence of said nucleic acid to be detected,
and subsequently detecting the amplified nucleic acid to be detected,
characterized in that
the sequence of said primer is selected from the sequences with SEQ ID Nos.: 1-28 or is complementary to said sequences, or is a fragment thereof or is complementary to said fragment or comprises any of said sequences or said complementary sequence. This embodiment of the invention is referred to just as amplification method hereinbelow. The term amplification method includes all preferred embodiments.

The skilled worker appreciates that it is possible, starting from the teaching of the present invention, also to design primers which slightly deviate from the primers of the invention but which nevertheless function. Conceivable primers are thus also those which, compared to the primers of the invention, are extended or truncated by at least one, two or three nucleotides at the 5′ and/or 3′ end. In particular it is possible for extensions or truncations at the 5′ end of the primers to provide still functional primers which may be used according to the invention. It is likewise conceivable that individual or a few nucleotides of a primer can be replaced with other nucleotides, as long as the specificity of said primer and the melting point of said primer are not altered too much.

It is obvious to the skilled worker that it is also possible to use, in addition to the usual nucleotides A, G, C and T, modified nucleotides such as inosine, etc. The teaching of the present invention provides for such modifications, starting from the subject matter of the claims.

Most preference is given to the amplification method of the invention if at least two primers have a sequence which is essentially a partial sequence of the nucleic acid to be detected, the sequences of said primers being selected from the sequences with SEQ ID Nos.: 1-28 or being complementary to said sequences, or is a fragment thereof or is complementary to said fragment or comprises any of said sequences or said complementary sequence.

Particular preference is given to the primer(s) having a sequence which is essentially a partial sequence of the nucleic acid to be detected, the sequences of said primers being selected from the sequences with SEQ ID Nos.: 1-13 or being complementary to said sequences, or is a fragment thereof or is complementary to said fragment or comprises any of said sequences or said complementary sequence. According to the invention, preference is given to the primers being labeled.

The explanations below apply both to the hybridization method of the invention and to the amplification method of the invention and also to the coupled amplification/hybridization method. Particular preference is given to coupled amplification/hybridization methods. In this case amplification is carried out using the sequence-specific primers of the invention and the amplification product obtained in this way is detected using the sequence-specific probes of the invention. This “multiplex approach” is very useful especially for identification and differentiation of bacteria.

Various reactions may be used as nucleic acid amplification reaction. Preference is given to using the polymerase chain reaction (PCR). The various embodiments of the PCR technique are known to the skilled worker, see, for example, Mullis (1990) Target amplification for DNA analysis by the polymerase chain reaction. Ann. Biol Chem (Paris) 48(8), 579-582. Further amplification techniques which may be applied are “nucleic acid strand-based amplification” (NASBA), “transcriptase mediated amplifcation” (TMA), “reverse transcriptase polymerase chain reaction” (RT-PCR), “Q-β replicase amplification” (β-Q-Replicase) and the “single strand displacement amplification” (SDA) . NASBA and other transcription-based amplification methods are discussed in Chan and Fox, Reviews in Medical Microbiology (1999), 10 (4), 185-196.

The simplest form of detecting the nucleic acid to be detected comprises cutting the amplicon specifically, for example by digestion with a restriction enzyme, and analyzing the ethidium bromide-stained fragments produced on an agarose gel. Hybridization systems are also very common. The hybridization is normally carried out by immobilizing either the composition containing the amplification product or a part thereof or the probe on a solid phase and contacting it with the in each case other hybridization partner. Possible solid phases are a large variety of materials, for example nylon, nitrocellulose, polystyrene, silicatic materials, etc. It is also conceivable to use a microtiter plate as solid phase. This may also involve the target sequence hybridizing with a capture probe in solution beforehand and then binding said capture probe to a solid phase.

Usually, amplification of the nucleic acid to be detected involves at least one labeled probe or at least one labeled primer.

In one embodiment of the method of the invention, the nucleic acid probes are immobilized on the solid phase and said solid phase is subsequently contacted with the composition containing the labeled nucleic acids to be detected or a part thereof. Preference is given to immobilizing at least two probes, more preferably at least five probes, even more preferably at least ten probes, on the solid phase. Different probes may be immobilized in different zones. By incubating the amplification product or the sample comprise the nucleic acid to be detected or a part thereof with a solid phase prepared in this way and containing immobilized probes, it is possible to obtain via a single hybridization step information about hybridization of said amplification product with all immobilized probes. Said solid phase is therefore preferably a microarray of immobilized probes on a solid phase. “DNA chips” of this kind allow a large number of different oligonucleotides to be immobilized on a small area. The solid phases suitable for DNA chips preferably comprise silicatic materials such as glass, etc. In this embodiment, the primer label is preferably a fluorescent label. After incubation with the amplification product or with the sample comprise the nucleic acid to be detected or a part thereof, it is possible to rapidly analyze the DNA chip using a scanning device. Such devices are known to the skilled worker. A review on chip technology can be found in McGlennen (2001) Miniaturization technologies for molecular diagnostics. Clin Chem 47(3), 393-402.

In this embodiment of the method of the invention the nucleic acid to be detected is labeled. A large variety of labels is possible here, such as fluorescent dyes, biotin or digoxigenin, for example.

Known fluorescent labels are fluorescein, FITC, cyanine dyes, rhodamines, Rhodamin₆₀₀R phycoerythrin, Texas Red, etc.

A radiolabel such as, for example, ¹²⁵I, ³⁵S, ³²p, ³⁵p is also conceivable.

Particle labeling, for example, with latex, is also conceivable. Such particles are usually dry, in the micron range and uniform.

The labels are normally covalently linked to the oligonucleotides. While a fluorescent label, for example, can be detected directly, biotin and digoxigenin labels may be detected after incubation with suitable binding molecules or conjugated partners. Examples of binding partners other than biotin/streptavidin are antigen/antibody systems, hapten/anti-hapten systems, biotin/avidin, folic acid/folate-binding proteins, complementary nucleic acids, proteins A, G and immunoglobulin, etc. (M.N. Bobrov, et al. J. Immunol. Methods, 125, 279, (1989)).

For example, it is possible to detect a biotin-labeled oligonucleotide by contacting it with a solution containing streptavidin coupled to an enzyme, said enzyme, for example, peroxidase or alkaline phosphatase, converting a substrate which produces a dye or results in chemiluminescence. Possible enzymes for this use purpose are hydrolases, lyases, oxido reductases, transferases, isomerases and ligases. Further examples are peroxidases, glucose oxidases, phosphatases, esterases and glycosidases. Methods of this kind are known per se to the skilled worker (Wetmur JG, Crit Rev Biochem Mol Biol 1991; (3-4): 227-59; Temsamani J. et al. Mol Biotechnol June 1996 ; 5(3): 223-32). In some methods in which enzymes act as conjugate partners color-changing substances must be present (Tijssen, P. Practice and Theory of Enzyme Immunoassays in Laboratory Techniques in Biochemistry and Molecular Biology, Edited by R. H. Burton and P. H. van Knippenberg (1998)).

Another preferred conjugate comprises an enzyme which is coupled to an antibody (Williams, J. Immunol. Methods, 79, 261 (1984)). It is furthermore common to label the nucleic acid to be detected with a gold-streptavidin conjugate, enabling a biotin-labeled oligonucleotide to be detected.

However, binding partners forming covalent bonds with one another, such as, for example, sulfhydryl-reactive groups such as maleimide and haloacetyl derivatives and amine-reactive groups such as isothiocyanates, succinimidyl esters and sulfonyl halides, are also conceivable.

If the nucleic acids to be detected are labeled, then the probes are usually unlabeled. The nucleic acids to be detected are labeled essentially according to methods described in the prior art (U.S. Pat. No. 6,037,127).

The label may be introduced into the nucleic acid to be detected by chemical or enzymic methods or by direct incorporation of labeled bases into said nucleic acid to be detected. In a preferred embodiment, sequences to be detected, which have incorporated labels, are produced by means of labeled bases or labeled primers during amplification of the nucleic acid to be detected. Labeled primers may be prepared by chemical synthesis, for example by means of the phosphoramidite method by substituting labeled phosphoramidite bases for bases of said primer during primer synthesis. As an alternative to this, it is possible to prepare primers containing modified bases to which labels are chemically bound after primer synthesis. Methods for labeling the nucleic acid to be detected, without amplifying said nucleic acid to be detected and/or providing it with a modification, are also possible. For example, ribosomal RNA species can specifically hybridize with a DNA probe and be detected as RNA/DNA hybrid, using an RNA/DNA-specific antibody.

Another possibility is introducing labels with the aid of T4 polynucleotide kinase or of a terminal transferase enzyme. Thus it is conceivable to introduce radioactive or fluorescent labels (Sambrook et al., Molecular Cloning; Cold Spring Harbor Laboratory Press, Vol. 2, 9.34-9.37 (1989); Cardullo et al. PNAS, 85, 8790; Morrison, Anal. Biochem, 174, 101 (1988).

Labels may be introduced at one or both ends of the nucleic acid sequence of the nucleic acid to be detected. Labels may also be introduced within the nucleic acid sequence of the nucleic acid to be detected. It is also possible to introduce a plurality of labels into a nucleic acid to be detected.

In a different embodiment, at least one of the probes has a label. The probes are labeled according to the same methods described in the prior art, as already illustrated above for the labeling of the nucleic acid to be detected. Usually, the composition comprising the amplification product or a part thereof is immobilized on a solid phase and contacted with a composition comprising at least one probe. In this embodiment too, preference is given to carrying out hybridization with more than one probe. For this purpose, a plurality of solid phases may be provided on which the amplification product or the sample comprising the nucleic acid to be detected is immobilized. However, it is also possible to immobilize a small amount of said amplification product on a solid phase at a plurality of spatially separated regions. These different spots are then contacted with in each case different probes (hybridization).

The present invention further relates to an apparatus for detecting periodontitis- and caries-associated bacteria, comprising a solid phase on which one or more sequence- and/or species— specific nucleic acid probes are immobilized, characterized in that said sequence- and/or species-specific nucleic acid probe is selected from the sequences with SEQ ID Nos.: 1-28 or. is complementary to said sequences, or is a fragment thereof or is complementary to said fragment or comprises any of said sequences or said complementary sequence. According to the invention, preference is given to the sequence-specific nucleic acid probe being selected from the sequences with SEQ ID Nos.: 14-28 or being complementary to said sequences or being a fragment thereof or being complementary to said fragment or comprising any of said sequences or said complementary sequence. When a plurality of oligonucleotides are immobilized, said oligonucleotides are present on the solid phase in a spatially separated manner. The solid phase is preferably designed in the form of a DNA chip.

The solid phase of the device of the invention may be a chromatographic material. Since the analyte is mainly hydrophilic, hydrophilic properties of said chromatographic material of the test strip are important for carrying out the method of the invention. Said chromatographic material may comprise inorganic powders such as silicatic materials, magnesium sulfate and aluminum, and may furthermore comprise synthetic or modified naturally occurring polymers such as nitrocellulose, cellulose acetate, cellulose, polyvinyl chloride or polyvinyl acetate, polyacrylamide, nylon, crosslinked dextran, agarose, polyacrylate, etc., and may furthermore comprise coated material such as ceramic materials and glass. Most preference is given to using nitrocellulose as chromatographic material. In addition, the introduction of positively charged ionic groups into nitrocellulose or nylon membranes, for example, may improve the hydrophilic properties of said chromatographic material.

The chromatographic material may be installed in a housing or the like. Said housing is usually water-insoluble, rigid and may comprise a multiplicity of organic and inorganic materials. It is important that the housing does not interfere with the capillary properties of the chromatographic material, that said housing does not bind test components unspecifically and that said housing does not interfere with the detection system.

Preference is given to the device of the invention if the sequence- and/or species-specific nucleic acid probes are bound via a linker to the solid phase of said device. The linker acts as a spacer between probe and membrane. In the present case, said linkers are usually polymers which extend the target sequence-complementary part of the probe at the 5′ or 3′ end but which themselves are noncoding. They may be base sequences of a noncoding nucleic acid structure or other polymeric units such as, for example, polyethers, polyesters, and the like. The nature of said linker must be such that the latter is not or only weakly adversely influenced the hybridization properties of the probe. This may be avoided by the absence of self-complementary structures. The chemical preconditions for irreversible coupling of the probe to the support material must also be present. A crucial requirement for proper functioning of the probe, in addition to its properties of forming a stable hybrid with the target sequence, is the chemistry of coupling to the surface. Chemical groups must be present which make irreversible binding possible with the immobilization techniques used. Said groups may be amine groups, thiol groups, carbamides, succinimides, and the like.

However, other possibilities of generating spacers or linkers between the probe and the membrane are also known to the skilled worker. Probe oligonucleotides may be bound, for example, via proteins to the membrane surface. The proteins charged with the probe may then be bound to the porous membrane according to standard methods. An example of standard methods is coupling via homobifunctional coupling reagents or heterobifunctional coupling reagents. Homobifunctional ones have identical reactive groups. These are typically amines and/or thiols. Thiols may be coupled synthetically directly to oligonucleotides and may react with cysteine residues, for example, under oxidative conditions to give disulfide bridges. For an amine-amine coupling, amines may be coupled as homobifunctional coupling reagents synthetically directly to oligonucleotides and bound via imidoesters or succinimide esters to the surface or the protein. Heterobifunctional coupling reagents have different reactive groups and allow coupling of various functional groups. Preference is given to the formation of amino-thiol couplings. A hetero-bifunctional coupling reagent which comprises both a succinimide ester maleimide or iodoacetamide may be used to couple thiolated oligonucleotides. Another important coupling reagent is carbodiimides which couple carbonyl radicals to amines. The most important representative here is 1-ethyl-3-(3-dimethylaminopropyl)carbodiimide (EDAC). Here it is possible to couple amino-modified oligonucleotides to membranes containing carbonyl radicals. In this chemistry, the coupling reagent is not incorporated into the compound.

The present invention further relates to a nucleic acid which is selected from the sequences with SEQ ID Nos.: 1-28 or which is complementary to said sequences or which is a fragment thereof or which is complementary to said fragment or which comprises any of said sequences or said complementary sequence. The present invention furthermore relates to a composition or to a kit for detecting periodontitis- and caries-associated bacteria, comprising one or more of the nucleic acids of the invention. The present invention in particular relates to a kit for amplifying a nucleic acid to be detected which is a fragment of the genome of a periodontitis- or caries-associated bacterium or is complementary to said fragment, which kit comprises one or more of the nucleic acids of the invention.

The kit comprises, in addition to the nucleic acids of the invention, all components required for amplification of the target sequence, such as primers, buffer systems, enzymes.

Most preference is given to a kit for amplifying a nucleic acid to be detected which is a fragment of the genome of a periodontitis- or caries-associated bacterium or is complementary to said fragment, which kit comprises one or more nucleic acids selected from the sequences with SEQ ID Nos.: 1-13 or complementary to said sequences, or a fragment thereof, or complementary to said fragment or comprising any of said sequences or said complementary sequence.

The invention also relates to the use of the device of the invention for detecting periodontitis- and caries-associated bacteria. The invention furthermore relates to the use of the nucleic acid of the invention or of the kit of the invention for detecting periodontitis- and caries-associated bacteria.

The nucleic acid to be detected may be present in any composition suspected of containing bacteria, in particular periodontopathogenic and caries-associated bacteria. It may be primary material, for example secretions, sulcus fluid, swabs and blood. It may be cultures of microorganisms already grown in liquid or solid media.

### DESCRIPTION OF THE FIGURES

FIG. 1: SEQ ID No.: 1-13

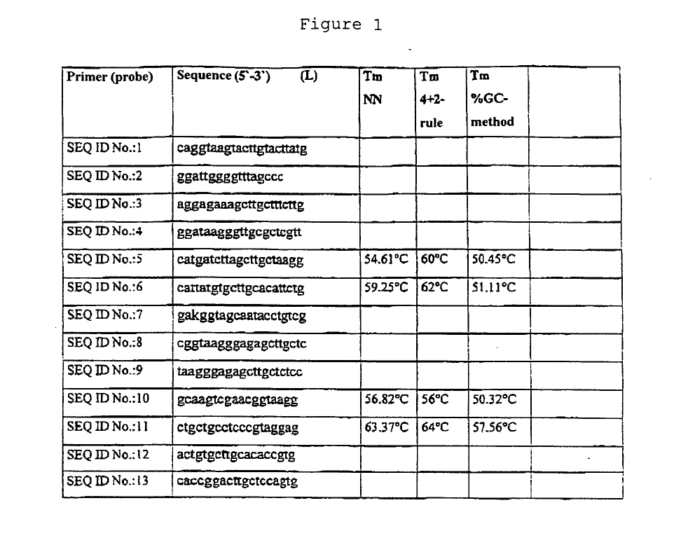

FIG. 2: SEQ ID No.: 14-28

FIG. 3, densitometric evaluation of a dot blot hybridization for determining the specificity of probes SEQ ID No: 15, 19, 21, 22, 25, 28, 27.

The abbreviations have the following meanings:
Aa=Acrinobacillus actinomycetemcomitans;
Pg=Porphyromonas gingivalis; Pi=Prevotella intermedia;
Bf=Bacteroides forsythus; Td=Treponema denticola;
Smut=Streptococcus mutans; Ssob=Streptococcus sobrinus;
E.col=Escherichia coli; hDNA=human DNA;
N-Kon=negative control (amplification without target nucleic acid).

The amplification products were applied to a membrane, as described in example 1, hybridized against the probes SEQ ID No: 15, 19, 21, 22, 25, 28, 27 and evaluated autoradiographically using a densitometer (Vilber Lourmat, Bio-Profil, Frobel Laborgerate, Lindau, Germany).

### EXAMPLES

### Example 1

DNA/RNA Isolation:

Bacterial nucleic acid was obtained either from solid nutrient media, liquid media or from primary material after appropriate pretreatment.

The following bacterial species were studied: Actinobacillus actinomycetemcomitans, Porphyromonas gingivalis, Prevotella intermedia, Bacteroides forsythus; Treponema denticola, Streptococcus mutans, Streptococcus sobrinus;

For this purpose, bacterial material was removed from solid media, using a sterile inoculation loop and suspended in 300 μl of 10 mM Tris/HCl pH 7.5. 1 ml was removed from liquid cultures, centrifuged in a bench centrifuge at 13 000 rpm for 5 min and, after discarding the supernatant, resuspended in 300 μl of 10 mM Tris/HCl pH 7.5. Primary material was removed from dental pockets using “paper points”. The swabs obtained in this way were incubated in a Thermomixer (Eppendorf, Hamburg, Germany) at 95° C. for 15 min, sonicated in an ultrasound bath (Bandelin) for 15 min and centrifuged in a bench centrifuge at 13 000 rpm for 10 min. In each case 5 μl of the supernatant were used in the amplification reaction.

Amplification:

All primers were commercially synthesized (Interactiva, Ulm, Germany). The primers used for amplifying target sequences of the abovementioned organisms were SEQ ID Nos. 1 to 13.

The PCR mixture contained 1× Taq buffer (Qiagen, Hilden, Germany), in each case 1 μM of primer, 200 μM dNTP (Roche) and 1 U of Hotstar Taq polymerase (Qiagen, Hilden, Germany). The PCR amplification was carried out on a Thermocycler PE 9600 (ABI, Weiterstadt, Germany), with 95° C. for 15 min, 10 cycles of 95° C. for 30 s and 60° C. for 2 min and 20 cycles of 95° C. for 10 s, 55° C. for 50 s and 70° C. for 30 s.

The NucliSens amplification kit (Organon Technika, Boxtel, Netherlands) was used according to the manufacturer's instructions for RNA amplification by the NASBA technique:
1. preparation of the amplification mix: 8 μl of “reagent sphere” dissolved in “reagent dilution” buffer (contains the enzymes required for the reaction), 5 μl of KCl solution, final concentration 70 mM KCl, and 2 μl of primer solution, final concentration 0.5 μM primer;
2. add 5 μl of RNA solution and incubate in a water bath at 41° C. for 60 min.

The DNA/RNA amplicon was detected with either an ethidium bromide-stained agarose gel or by hybridization.

Detection of Amplicons by Probe Hybridization:

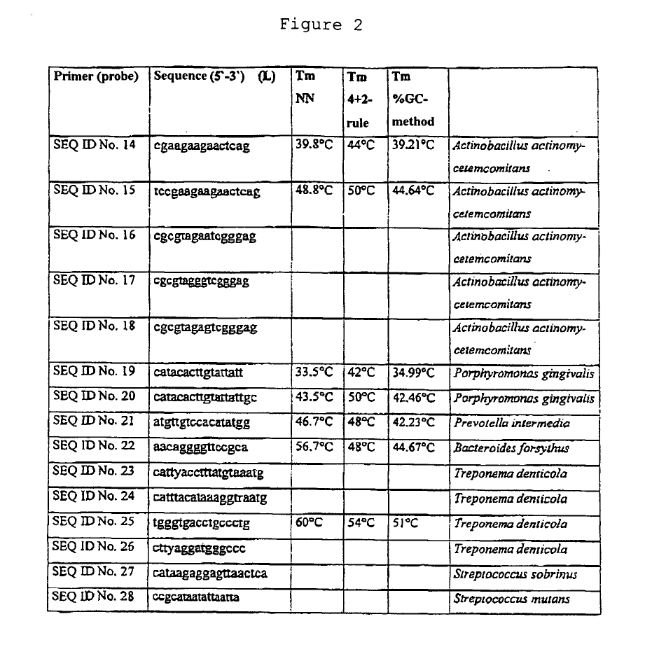

All probes were biotinylated at the 5′ end, in order to be able to detect target sequence/probe hybrids via reporter enzymes coupled to streptavidin. The probes used are oligonucleotides having the sequences SEQ ID NO 14 to 28 (see FIG. 2).

Blotting paper (Blotting Papier GB002, Schleicher & Schull, Dassel, Germany) and a nylon membrane (Biodyne A, Pall, Portsmouth, England) were cut to the size of the blotting apparatus (Minifold Schleicher & Schull, Dassel, Germany) and soaked with 10×SSC. 250 μl of denaturing solution (50 mM NaOH; 1.5 M NaCl) were initially introduced into the openings of the assembled apparatus and 20 μl of amplicon were added by pipetting. After applying a vacuum, all of the fluid was allowed to soak through completely. This was followed by rinsing with 10×SSC buffer. After drying to completion, the membrane was fixed in a UV crosslinker (UV Stratalinker 2400, Stratagene, La Jolla, USA) at 1200 Joule/cm² and washed with distilled water and dried.

All hybridizations were carried out in glass tubes in a hybridization oven at 45° C. (Hybaid Mini Oven MkII, MWG-Biotech, Ebersberg, Germany). The membrane coated with DNA/RNA amplicon was rolled in in a dry state and added to a glass tube. The membrane was then incubated with constant rotating with prewarmed hybridization buffer for 5 min. After adding 2 pmol of biotinylated probe, the hybridization reaction was performed for one hour. Unbound or only partially bound probe was removed by 30 min of incubation with stringent buffer at 45° C., with one exchange of said prewarmed stringent buffer. This was followed by adding blocking reagent and further incubation at 37° C. for 15 min. The hybrids were detected via a streptavidin-alkaline phosphatase conjugate either calorimetrically by adding NBT/BCIP or autoradiographically by spraying on chemiluminescent substrate (Lumi-Phos 530, Cellmark Diagnostics, Abindon, England). For this purpose, streptavidin-alkaline phosphatase conjugate was added and incubated at 37° C. for 30 min. The membrane was then washed twice with substrate buffer for 15 min each. The membrane was then removed, Lumi-Phos reagent was sprayed on, followed by exposing an X-ray film for 2 h. As an alternative to this, substrate buffer containing NBT/BCIP was added, waiting for the color to develop.

Solutions Used:

10×SSC solution (standard saline citrate):
1.5M NaCl, 0.15M trisodium citrate;
   Hybridization Buffer:
7% SDS (sodium dodecyl sulfate), 0.25M phosphate buffer pH 7.5;
   Stringent Washing Solution (Stringent Buffer):
3 M TMCL (tetramethylammonium chloride), 50 mM Tris/Cl, 2 mM EDTA, 0.1% SDS;
   Solution for Saturating Membrane Binding Sites:
5 g/l blocking reagent (Roche) in maleic acid buffer pH 7.5 (4.13 g of NaCl and 5.53 g of maleic acid in 500 ml of water, pH adjusted to 7.5 with 5 M NaOH);
   Substrate Buffer:
274 mM Tris/Cl pH 7.5, 68.6 mM Na₃ citrate, 200 mM NaCl, 27.4 mM MgCl₂*6 H₂O;
   BCIP:
50 mg/ml 5-bromo-4-chloro-3-indonyl phosphate toluidinium salt, in 100% dimethylformamide;
   NBT:
75 mg/ml Nitro Blue tetrazolium salt in 70% dimethyl-formamide;

The autoradiograms were evaluated densitometrically. The 100% base value used was the amplicon dot of the species from which the probe sequence had been derived. Controls which were always co-applied as dots to the membrane were a sample to which water rather than nucleic acid solution had been added and a sample containing 100 ng of isolated human DNA.

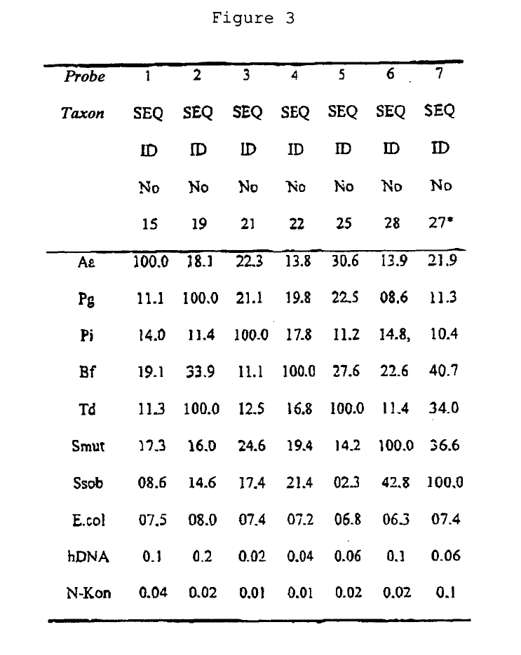

FIG. 3 depicts the results of example 1. The % values of the densitometric evaluation are indicated. The value of the probe homologous to the species was set to 100%. The methods described here may be used for identifying and differentiating the corresponding bacteria either from primary material (e.g. dental swabs, blood, and the like) or from bacterial liquid or solid media.

1. A method for detecting periodontitis- and caries-associated bacteria, which comprises hybridizing a nucleic acid to be detected which is a fragment of the genome of a periodontitis- or caries-associated bacterium or is complementary to said fragment to a sequence- and/or species-specific nucleic acid probe and subsequently detecting said nucleic acid to be detected or said hybridization of said nucleic acid to be detected to said sequence-specific nucleic acid probe, characterized in that
said sequence-specific nucleic acid probe is selected from the sequences with SEQ ID Nos.: 1-28 or is complementary to said sequences, or is a fragment thereof or is complementary to said fragment or comprises any of said sequences or said complementary sequence.

2. The method as claimed in claim 1, characterized in that the sequence-specific nucleic acid probe is selected from the sequences with SEQ ID Nos.: 14-28 or is complementary to said sequences, or is a fragment thereof or is complementary to said fragment or comprises any of said sequences or said complementary sequence.

3. The method as claimed in claim 1, characterized in that the nucleic acid to be detected is an amplification product, said amplification having been carried out using sequence-specific amplification primers.

4. The method as claimed in any of claim 1, characterized in that the nucleic acid to be detected is an amplification product, at least one amplification primer being selected from the sequences with SEQ ID Nos.: 1-28 or being complementary to said sequences, or is a fragment thereof or is complementary to said fragment or comprises any of said sequences or said complementary sequence.

5. The method as claimed in claim 4, characterized in that at least one amplification primer is selected from the sequences with SEQ ID Nos.: 1-13 or is complementary to said sequences, or is a fragment thereof or is complementary to said fragment or comprises any of said sequences or said complementary sequence.

6. The method as claimed claim 1, characterized in that the nucleic acid probes are immobilized.

7. The method claim 6, characterized in that the nucleic acid to be detected is labeled.

8. The method as claimed in claim 1, characterized in that the nucleic acid probes are labeled and the nucleic acid to be detected is immobilized.

9. A method for detecting periodontitis- and caries-associated bacteria, which comprises
amplifying a nucleic acid to be detected which is a fragment of the genome of a periodontitis- or caries-associated bacterium or is complementary to said fragment, said amplification being carried out using primers at least one of which has a sequence which is essentially a partial sequence of said nucleic acid to be detected,
and subsequently detecting the amplified nucleic acid to be detected, characterized in that
the sequence of said primer is selected from the sequences with SEQ ID Nos.: 1-28 or is complementary to said sequences, or is a fragment thereof or is complementary to said fragment or comprises any of said sequences or said complementary sequence.

10. The method as claimed in claim 9, characterized in that at least two primers have a sequence which is essentially a partial sequence of the nucleic acid to be detected, the sequences of said primers being selected from the sequences with SEQ ID Nos.: 1-28 or being complementary to said sequences, or is a fragment thereof or is complementary to said fragment or comprises any of said sequences or said complementary sequence.

11. The method as claimed in claim 9, characterized in that the primer/primers has/have a sequence which is essentially a partial sequence of the nucleic acid to be detected, the sequences of said primers being selected from the sequences with SEQ ID Nos.: 1-13 or being complementary to said sequences, or is a fragment thereof or is complementary to said fragment or comprises any of said sequences or said complementary sequence.

12. The method as claimed in claim 9, characterized in that amplification is carried out using the polymerase chain reaction.

13. The method as claimed in claim 9, characterized in that the primers are labeled.

14. An apparatus for detecting periodontitis- and caries-associated bacteria, comprising a solid phase on which one or more sequence- and/or species-specific nucleic acid probes have been immobilized, characterized in that said sequence-specific nucleic acid probe is selected from the sequences with SEQ ID Nos.: 1-28 or is complementary to said sequences, or is a fragment thereof or is complementary to said fragment or comprises any of said sequences or said complementary sequence.

15. The apparatus as claimed in claim 14, characterized in that the sequence-specific nucleic acid probe is selected from the sequences with SEQ ID Nos.: 14-28 or is complementary to said sequences, or is a fragment thereof or is complementary to said fragment or comprises any of said sequences or said complementary sequence.

16. The apparatus as claimed in claim 14, characterized in that the sequence- and/or species-specific nucleic acid probes is bound via a linker to the solid phase of said apparatus.

17. A nucleic acid selected from the sequences with SEQ ID Nos.: 1-28 or complementary to said sequences, or a fragment thereof, or complementary to said fragment or comprising any of said sequences or said complementary sequence.

18. A kit for detecting periodontitis- and caries-associated bacteria, comprising one or more nucleic acids as claimed in claim 17.

19. A kit for amplifying a nucleic acid to be detected which is a fragment of the genome of a periodontitis- or caries-associated bacterium or is complementary to said fragment, which kit comprises one or more nucleic acids as claimed in claim 17.

20. A kit for amplifying a nucleic acid to be detected which is a fragment of the genome of a periodontitis- or caries-associated bacterium or is complementary to said fragment, which kit comprises one or more nucleic acids selected from the sequences with SEQ ID Nos.: 1-13 or complementary to said sequences, or a fragment thereof, or complementary to said fragment or comprising any of said sequences or said complementary sequence.

21. The use of the apparatus as claimed in claim 14 for detecting periodontitis- and caries-associated bacteria.

22. The use of the nucleic acid as claimed in claim 17 or of the kit as claimed in claim 18 for detecting periodontitis- and caries- associated bacteria.
